(19) United States
(12) Patent Application Publication
(10) Pub. No.: US 2008/0146549 A1
(43) Pub. Date: Jun. 19, 2008

(54) Accelerated opiate dependence detoxification process

(76) Inventor: Peter R. Coleman, Richmond, VA (US)

Correspondence Address: TROUTMAN SANDERS LLP, 600 PEACHTREE STREET , NE, ATLANTA, GA, 30308, US

(21) Appl. No.: 12/002,670

(22) Filed: Dec. 18, 2007

Related U.S. Application Data
(60) Provisional application No. 60/875,437, filed on Dec. 18, 2006.

(51) Int. Cl. A61K 31/55; A61K 31/4168; A61K 31/495; A61K 31/4355; A61P 25/36

(52) U.S. Cl. 514/221; 514/401; 514/256; 514/282

(57) ABSTRACT

The present invention provides accelerated detoxification methods for the treatment of a substance abuse-related condition in a subject. A method of the present invention may comprise administering to the subject an effective amount of at least one sedative (e.g, clonidine, diazepam, or olanzapine) and a micro-dose of an opioid antagonist (e.g., naltrexone or naloxone) for at least one day; optionally administering a small dose of an opiate, and administering to the subject a detoxifying amount of a second opioid antagonist (e.g., naloxone); and may further comprise administering to the subject a third opioid antagonist (such as, naltrexone) for an extended period of time (e.g., 12 months).

### RELATED APPLICATIONS

This application claims the priority of U.S. Provisional Patent Application Ser. No. 60/875,437, filed Dec. 18, 2006, which is hereby incorporated in its entirety.

### TECHNICAL FIELD

The invention relates to methods and pharmaceutical compositions for the treatment of substance abuse-related conditions, particularly opiate dependence.

### BACKGROUND OF THE INVENTION

Opioids have been used for centuries for their analgesic, pleasurable effects, and for the treatment of certain clinical conditions. However, these beneficial effects have been accompanied by the problems of substance abuse, dependence, and addiction. It is estimated that the medical care costs associated with heroin addiction alone in the United States exceeds five billion dollars, and this does not include the associated social costs. Other studies estimate that there were about 100,000 to 120,000 emergency department visits in the United States in 2002 that were related to the uses of heroin and prescription opiates.

Various approaches have been tried for the treatment of opioid dependence, such as, drug-free, abstinence-based treatment, and opiate agonist treatment. Agonist-based treatment programs substitute one addictive opiate for another, and many patients find this an unsatisfactory solution. These patients want to be free from drugs and the problems associated with their use. Abstinence based programs suffer from two problems. Firstly, detoxification is difficult and often unsuccessful. Secondly, patients find it difficult to stay abstinent. Inpatient and outpatient detoxification treatments that use supportive medications are often found to be expensive, uncomfortable, and are often not completed. There exists a need for a system and method for effective, cost-efficient, and rapid detoxifying for the treatment of opioid abuse and dependence. There is also a need for a treatment that helps patients remain abstinent after they are detoxified.

### SUMMARY OF THE INVENTION

The present invention provides a method for treating a substance abuse-related condition, including, without limitation, a substance withdrawal syndrome, in a subject, comprising the steps of: (a) administering to the subject an effective amount of at least one sedative or other symptomatic medicine and optionally a micro-dose of an opioid antagonist (a first antagonist) for at least one day; and (b) administering to the subject, a detoxifying amount of the same or a different opioid antagonist (a second opioid antagonist); and may further comprise administering to the subject the same or a different opioid antagonist (a third opioid antagonist) for an extended period of time. The method may further comprise a step of evaluating a subject with a substance abuse-related condition prior to step (a), or at any time in the method.

In an aspect of the invention, the at least one sedative may comprise, diazepam, olanzapine, or other sedatives, or a pharmaceutically acceptable salt or complex thereof. In another aspect, the method of the present invention may further comprise administering to the subject a symptomatic medicine such as clonidine or another alpha-agonist, a compound with sedating qualities, or similar medicine that reduces opiate withdrawal symptoms, such as but not limited to, tiagabine. In another aspect, the method of the present invention may further comprise administering to the subject an effective amount of at least one opioid before or concurrent with step (a), where the at least one opioid may comprise tramadol, buprenorphine, methadone, or other known opioid, or a pharmaceutically acceptable salt or complex thereof. In yet another aspect, the method of the present invention may further comprise administering to the subject an effective amount of octreotide, phenobarbital, chlorpromazine, or pharmaceutically acceptable salts or complexes thereof before step (b), including, without limitation, immediately before the performance of step (b).

The first opioid antagonist, a second opioid antagonist, and a third opioid antagonist may comprise the same or different opioid antagonists, or a combination of opioid antagonists. In one aspect, a first opioid antagonist may comprise naltrexone, naloxone, or a pharmaceutically acceptable salt or complex thereof. In another aspect, a second opioid antagonist may comprise naloxone, naltrexone, or a pharmaceutically acceptable salt or complex thereof. In yet another aspect, a third opioid antagonist may comprise naltrexone, or another opioid antagonist, or a pharmaceutically acceptable salt or complex thereof. A third opioid antagonist may be in the form of a controlled release pharmaceutical composition, where it may release a third opioid antagonist over a period of about 6 to about 8 weeks. In still another aspect, the subject may be treated with the third opioid antagonist for about 3, 4, 5, 6, 7, 8, 9, 10, 11 or 12 months. A method of the present invention comprises administration of an implant device comprising an opioid antagonist, that releases the third opioid antagonist at zero order kinetics. Such an implant device may remain implanted for up to 1 month, 2 months, 3 months, 4 months, 5 months, 6 months, 7 months, 8 months, 9 months, 10 months, 11 months, or up to 12 months. The implant device may be removed and a new device containing the same or a different opioid antagonist at the same or a different release rate is implanted.

Other features and advantages of the present invention will become apparent from the following detailed description. It should be understood, however, that the detailed description and the specific examples, while indicating aspects of the present invention, are given by way of illustration only, since various changes and modifications within the spirit and scope of the present invention will become apparent to those skilled in the art from this detailed description.

### DETAILED DESCRIPTION OF THE INVENTION

As used herein and in the appended claims, the singular forms “a,” “an,” and “the” include plural references, unless the content clearly dictates otherwise. Thus, for example, reference to “an opioid” includes a plurality of such opioids and equivalents thereof known to those skilled in the art, and reference to “the opioid antagonist” is a reference to one or more such opioid antagonists and equivalents thereof known to those skilled in the art, and so forth. All terms used herein are considered to be interpreted in their normally accepted usage by those skilled in the art. All publications, patent applications, patents, and other references mentioned herein are incorporated by reference in their entirety.

The present invention generally comprises accelerated, effective, and cost-efficient methods for the treatment of at least one substance abuse-related condition, including but not limited to, opiate dependence. Prior to the present invention, methods for detoxifying opiate-dependent persons required extensive sedation, such as general anesthesia, with high dosage amounts of an antagonist and the attendant high risk to the patients with such a drastic process and lengthy anesthesia. Such detoxification methods are required to be in a hospital setting for the safety of the subject undergoing the treatment. An alternative approach was to allow the subject to undergo withdrawal, with all of its negative, debilitating and unwanted side effects, to a point where an antagonist could be provided at doses lower than those which require hospitalization or general anesthesia. These prior methods comprise risky procedures or extremely uncomfortable procedures and are not desired by subjects. In contrast, the current invention provides relief from withdrawal symptoms during treatment; initiates withdrawal less drastically; the treatment may be provided in a physician's office in a substantially outpatient setting; there is no need for general anesthesia, and provides micro-doses of antagonist. Performance of the methods of the present invention in a physician's office allows for the subject to remain at home, receive medications and be treated in a comfortable and relaxed setting, and to return home soon after treatment on the summit day. Not only are these attributes important for the subject and his/her response to therapy, but they are also a significant cost savings for the subject and society.

The terms “substance abuse,” “substance-dependence,” “substance addiction,” “drug abuse,” “drug dependence,” and “drug addiction,” as used herein, may be used interchangeably and refer to the overindulgence in and dependence on an opiate, a stimulant, a depressant, and/or other chemical substance, leading to effects or conditions that are detrimental to the user's physical or mental health, and/or the welfare of others. Similarly, a substance abuse, dependence, or addiction-related condition may include, without limitation, any physical, mental, physiological, pathological, psychological, psychiatric, and/or clinical conditions related to the use or abstinence from use of a chemical substance, including any withdrawal syndrome resulting from discontinuation of use of substances, such as, opioids or opiates, either natural or synthetic. Typical opioid withdrawal syndrome may include, without limitation, sweating, restlessness, bone and/or joint aches, muscle spasms/twitching, runny nose or tearing, gastrointestinal conditions (e.g., without limitation, stomach cramps, nausea, vomiting, loose stool, and diarrhea), tremor, yawning, anxiety, and gooseflesh skin. As used herein, the term “substance” may include any natural, synthetic, opiate, opioid, or narcotic compounds or compositions (including, without limitation, opioid, opioid derivatives, opioid agonists, and opioid antagonists). Examples of such substances include, without limitation, opium, morphine, heroin, pethidine, methadone, buprenorphine, butorphanol, codeine, fentanyl, hydrocodone, hydromorphone, levorphanol, meperidine, oxycodone, pentazocine, propoxyphene, and tramadol.

As used herein, the phrase “treatment of a substance abuse-related condition” includes efforts, actions, conducts, and/or procedures to ameliorate, reduce, minimize, eliminate, or prevent any physical, mental, physiological, pathological, psychological, psychiatric, and/or clinical conditions or impairments related to or resulting from a substance abuse, addiction, dependence, or the withdrawal therefrom. For example, without limitation, clinical impairments or symptoms of a substance abuse-related condition may be ameliorated or minimized by diminishing any pain or discomfort suffered by the subject; by inhibiting, reducing, or preventing the development of a substance abuse-related condition; or by limiting, suspending, terminating, or otherwise controlling a substance abuse-related condition.

The present invention comprises a method for the treatment of a substance abuse-related condition in a subject, including the steps of: (a) administering to the subject an effective amount of at least one sedative and optionally a micro-dose of an opioid antagonist for at least one day and/or optionally a small dose of an opiate; and (b) administering to the subject a detoxifying amount of a second opioid antagonist. The method may further comprise repeating step (a) for multiple days, for example when detoxifying of subjects who are addicted to particular drugs is contemplated. The method may comprise administering to the subject a medication that relieves opioid withdrawal symptoms such as clonidine or tiagabine (Gabatril) for an extended period of time.

The method may further comprise (c) administering to the subject a third opioid antagonist for an extended period of time. The method may further comprise evaluating a subject with a substance abuse-related condition; prior to step (a). In one aspect, the method of the present invention may further include a step of administering to the subject an effective amount of at least one opioid (e.g., without limitation, tramadol, buprenorphine, methadone, or a pharmaceutically acceptable salt or complex thereof) before or concurrent with step (a). The at least one opioid may be administered to the subject, for example, for the entire treatment period, for example, without limitation, for eight days.

The term “an effective amount of at least one sedative,” as used herein, may include any amount of sedatives which may facilitate and/or improve the treatment of a substance abuse-related condition. Sedatives are well known in the art. Examples of suitable sedatives may include, without limitation, antipsychotics, atypical antipsychotics, alpidem, amobarbital, antihistamines, barbiturates, benzodiazepines, chloral hydrate, chlorazepate, chlordiazepoxide, clonazepam, clonidine, diazepam, diethyl ether, dimenhydrinate, diphenhydramine, doxylamine, ethchlorvynol, flunitrazepam, gamma-hydroxybutyrate, glutethimide, herbal sedatives, imidazopyridines, kava, lorazepam, meprobamate, methaqualone, methyl trichloride, methyprylon, olanzapine, phenabarbitol, pentobarbital, promethazine, pyrazolopyrimidines, seroquel, secobarbital, tiagabine, tranquilers, zaleplon, zolpidem, a pharmaceutically acceptable salt or complex thereof, a combination thereof, and a pharmaceutical composition comprising the same. In one aspect, the sedative may be clonidine, diazepam, or olanzapine. As used herein, the term “subject,” or “patient,” may include an animal, such as, without limitation, a human.

A method of the present invention comprises treatment for opiate addiction comprising, (a) administering on a first day of treatment an effective amount of a sedative to a subject undergoing treatment for opiate addiction; (b) optionally, administering on the first day of treatment an effective amount of an opiate to the subject undergoing treatment for opiate addiction; (c) administering on a day of treatment following the first day, an effective amount of a sedative and a micro-dose of an opioid antagonist; optionally, administering an effective amount of an opiate to the subject; (d) administering on a summit day of treatment, an effective amount of a opioid antagonist sufficient to bind to an effective number of opioid receptors of the subject, referred herein as a detoxifying amount of an opioid antagonist, and the binding of the effective amount of the antagonist to an effective number of opioid receptors effects detoxification.

A method of the present invention comprises treatment for opiate addiction comprising, (a) administering on the first day of treatment an effective amount of a sedative to a subject undergoing treatment for opiate addiction; (b) optionally, administering on the first day of treatment an effective amount of an opiate to the subject undergoing treatment for opiate addiction; optionally, administering a micro-dose of an opioid antagonist; (c) administering on a second day of treatment, an effective amount of a sedative and a micro-dose of an opioid antagonist, and optionally, an effective of amount of an opiate; (d) repeating step (c) so that the days of treatment equal 2, 3, 4, 5, 6, 7, 8, 9, 10, 11, 12, 13, 14, 15, 16, 17, 18, 19 or 20; (e) administering on a day of treatment, an effective amount of a opioid antagonist sufficient to bind to an effective number of opioid receptors of the subject, and optionally, (f) implanting, for long-term administration of an antagonist, or providing a third opioid antagonist for long term control of sobriety. Step (e) may be the last day of treatment, or may occur on the day prior to or the same day as step (f). Step (e) is referred to herein as the summit day. A third opioid antagonist may be provided orally, for example daily for 1-2 weeks, and may be followed by implantation of a slow-release antagonist delivery dosage form.

A method of the present invention comprises treatment for opiate addiction comprising, (a) administering on the first day of treatment to a subject undergoing treatment for opiate addiction, an effective amount of a sedative, an effective amount of an opiate and an effective amount of a micro-dose of an opiate antagonist, (b) repeating step (a) for several days, such as 2 days, 3 days, 4 days, 5 days, 6 days, or 7 days or up to 20 days; (c) administering on a day of treatment, the summit day, an effective amount of an opioid antagonist sufficient to bind to an effective number of opioid receptors of the subject, and optionally, (d) implanting or providing a third opioid antagonist for long-term administration of the antagonist. Step (d) may occur on the summit day or the day after the summit day. An alternative method comprises administering (a) for one or more days of treatment, followed by administering an effective amount of a sedative and an effective amount of a micro-dose of an opiate antagonist for one or more days until the summit day. The summit day is the day in which an effective amount of an opioid antagonist sufficient to bind to an effective number of opioid receptors of the subject. An oral dose of an antagonist may be given for a time period prior to providing an implant for longer term dosage of an antagonist.

Methods of the present invention comprise detoxifying subjects who are addicted to opiates and controlling the level of withdrawal symptoms experienced by the subjects while undergoing detoxification. In one aspect, subjects are evaluated for treatment, including the level of tolerance of withdrawal symptoms. Usually subjects may tolerate 18-24 hours of withdrawal. If needed, medications may be prescribed to lessen the effects of the withdrawal, such as without limitation, clonidine 0.1 mg every six hours and 0.1-0.2 mg at bedtime. Medications which may be given for decreasing withdrawal symptoms, include, but are not limited to, clonidine, tramadol (about 50-150 mg), benzodiazepines, such as diazepam (about 10-40 mg), antipsychotics or atypical antipsychotics, such as olanzipine (about 5 mg), phenobarbital (about 65 mg), or thorazine (about 100 mg). The subject may be evaluated, e.g., by a physician, among others, about 10-120 minutes, or about 30-60 minutes following the medications to examine whether any withdrawal symptoms have been reduced, and whether other problems develop. Small doses of opiates may be provided to the subject to lessen the withdrawal symptoms. An effective amount of an opiate is an amount that will lessen the withdrawal symptoms but is not an addicting amount. For example, 50 to 150 mg of Ultram may be administered three times daily, to lessen withdrawal symptoms.

The first opioid antagonist, the second opioid antagonist, and the third opioid antagonist used in the process of the present invention may be the same opioid antagonist, a different opioid antagonist, or a combination of opioid antagonists. In one aspect, the first opioid antagonist may comprise naltrexone, naloxone, or nalmefene, a pharmaceutically acceptable salt or complex thereof, a combination thereof, or a pharmaceutical composition comprising the same. In another aspect, the second opioid antagonist may comprise naloxone, a pharmaceutically acceptable salt or complex thereof, a combination thereof, or a pharmaceutical composition comprising the same. In yet another aspect, the third opioid antagonist may comprise naltrexone or a pharmaceutically acceptable salt or complex thereof, which may include any suitable pharmaceutical composition comprising naltrexone.

In one aspect, a micro-dose of opioid antagonist may be given to the subject during the treatment method, but before the summit day of the treatment. As used here, the term “micro-dose” refers to a small dose of an opioid antagonist, and it is a dose that is an effective amount to induce an amount of withdrawal symptoms that are uncomfortable, but acceptable to the subject undergoing treatment methods. The micro-dose administered may remain the same dose throughout the days of treatment, or may be administered in increasing doses. For example, but not limited to, a micro-dose may comprise 25 to 200 μg of naltrexone or some other opioid antagonist. The micro-dose opioid antagonist may be given intramuscularly, subcutaneously, orally or by some other route of administration. An example of a method of the present invention comprises administering on a first and second day an effective amount of a micro-dose of an opioid antagonist of about 50 to 100 μg, such as providing naltrexone in a dose of 50 to 100 μg. In another treatment method, an increasing amount of a micro-dose is administered over a time period of an eight day treatment regimen, from day two to day seven, wherein the beginning micro-dose is 25 μg and increases to about 125 μg or 150 μg by day seven. Examples of suitable opioid antagonists may include, without limitation, 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, naltrindole, or any other opioid antagonist. In one aspect, the opioid antagonist may be naloxone or naltrexone. The micro-dose, for example, without limitation, naltrexone, may be administered via routes that are suitable for the subject, including, but not limited to intramuscular, subcutaneous, oral, nasal, and mucosal routes. Aspects of the invention comprise giving micro-doses over a period of time as the subject is able to tolerate the antagonist and the withdrawal symptoms. Such micro-doses are administered prior to the summit day of treatment. As used herein, the term “summit day” refers to the day when a detoxifying amount of an opioid antagonist is given to a subject.

Various procedures may be performed during the summit day before a detoxifying amount of an opioid antagonist is given to a subject. In one aspect, medications such as phenobarbital (about 65 mg) and thorazine (about 100 mg) as well as Sandostatin (octreotide, about 100 μg) may be given to the subject. Any sedatives that are safe in an outpatient setting may be given. Factors, such as, the patient's age, weight, and/or reaction to a medication, may be considered in order to determine the dosage of the medications. Such practice is well known in the art

As used herein, the term “detoxifying amount of an opioid antagonist” includes an effective amount of an opioid antagonist which may substantially saturate, bind to, or block an effective number of the opioid receptors in a subject. The terms “substantially saturate” and “substantially block” an effective number of opioid receptors include about 75%, about 80%, about 85%, about 90%, about 95%, or higher, saturation or blockage of the opioid receptors in a subject.

In one aspect, a detoxifying amount comprises up to about 2000 micrograms of naloxone, which may be given, for example, intravenously or intramuscularly, to a subject over a period of time, such as, within 4-6 hours. Such a detoxifying amount of naloxone may be followed by about 0.5 mg to about 10 mg bolus administration of an antagonist such as naltrexone, or about 1 mg to about 8 mg, or about 2 mg to about 6 mg, or about 3 mg to about 5 mg, or about 1 mg, 2 mg, 3 mg, 4 mg, 5 mg, 6 mg, 7 mg 8 mg, 9 mg or 10 mg of an antagonist. Not wishing to be bound by any particular theory, it is currently thought that providing a bolus administration of an opiate antagonist provides antagonist support in the administration of an implant of an opiate antagonist, and may be additional support for the detoxifying amount of antagonist provided.

The treatment method days, up to but not including the summit day wherein a detoxifying amount of an opiate antagonist is provided, may be about 1 to about 15 days, or about 2 to about 8 days. On a summit day, a detoxifying amount such as 2,000 μg of an opiate antagonist such as naltrexone or naloxone may be administered intravenously, and optionally followed by a bolus administration of an opiate antagonist. The summit day may be followed by continuous administration of an opiate antagonist, such as by providing a sustained release delivery form of an opiate antagonist, such as naltrexone.

For example, an 8 day treatment method may be appropriate for subjects dependent on methadone, buprenorphine, suboxone, or other long-acting opiates because of the long half-life of those drugs and the severity of the withdrawal symptoms if the detoxifying treatment is performed too rapidly. In one aspect, the subject may use as small amounts of opioids (e.g., tramadol) as possible and use as large amounts of micro-dose opioid antagonists (e.g., naltrexone) as possible, in part to control withdrawal symptoms, during the treatment period up to the summit day. In the 8 day treatment method, the extent of the withdrawal symptoms of the subject are maintained at a tolerable point by the administration of sedatives and other actives, by providing small amounts of opioids, and by providing micro-doses of opioid antagonist. For example, on day 1, the subject is administered at least a sedative and a micro-dose of an opioid antagonist in amounts that are tolerated by the subject, optionally, a small amount of an opioid is administered. On days 2-7, at least the sedative is administered, and the amount of opioid antagonist is increased, such as in an increasing step-wise manner. Optionally, a small amount of an opioid is administered each day, or as needed. On day 8 (the summit day), the subject is administered a detoxifying amount of an opioid antagonist, such as by providing 2,000 μg of an opioid antagonist slowly, and this may be followed by a bolus administration of 1-10 mg of an opioid antagonist. On day 8 or 9, the subject may have a continuous release implant inserted in the subject's body, comprising an opioid antagonist, or the subject may be administered a daily dose regimen of an opiate antagonist for a time, and an implant may be provided afterward.

To aid in preventing relapse following the detoxifying treatment, a third opioid antagonist (e.g., naltrexone or Vivitrol) may be administered to the subject for an extended period of time, such as, for about 1 to about 24 months, or for about 12 months. The opioid antagonist may be given to the subject using any suitable method, such as, but not limited to, orally or in an implant form. In one aspect, the opioid antagonist, such as, naltrexone, may be given to the subject in the form of a controlled/sustained release pharmaceutical composition. For example, a sustained release pellet containing naltrexone may be implanted under the subject's skin, such as, without limitation, in the fatty tissue of the lower abdomen. The controlled release pharmaceutical composition may release an effective amount of the opioid antagonists (e.g., naltrexone) over a period of more than about 6 to about 8 weeks or more than about 8 to about 10 weeks, up to about 12 or about 24 months. Controlled release pharmaceutical compositions comprising opioid antagonist (e.g., naltrexone) and methods for delivering an effective amount of opioid antagonist are known in the art, see, e.g., United States Patent Publication No. 2002/0034534, the content of which is herein incorporated by reference in its entirety.

The compositions of the present invention may be administered to a subject independently, or together with other pharmaceutical compositions. It may also be administered to a subject together with food or a beverage.

A pharmaceutical composition of the present invention may be administered to a subject by known procedures, including, without limitation, oral administration, parenteral administration, transdermal administration, nasal administration, and by way of catheter. For example, the pharmaceutical composition may be administered orally, parenterally, by epifascial, intracapsular, intracranial, intracutaneous, intrathecal, intranasal, intramuscular, intraorbital, intraperitoneal, intraspinal, intrasternal, intravascular, intravenous, parenchymatous, subcutaneous, or sublingual administration. The pharmaceutical composition may be provided in an amount effective to treat a pathological or psychiatric condition in a subject to whom the composition is administered. As used herein, the phrase “effective to treat a disorder” means effective to eliminate, ameliorate, reduce, minimize, or prevent the clinical impairment or symptoms associated with the disorder.

For oral administration, the pharmaceutical composition of the present invention may be presented as capsules, tablets, powders, granules, or as a suspension, among others. The formulation may have conventional additives, such as, but not limited to, lactose, mannitol, corn starch, or potato starch. The formulation also may be presented with binders, such as, crystalline cellulose, cellulose derivatives, acacia, corn starch, and gelatins, among others. Additionally, the formulation may be presented with disintegrators, such as, but not limited to, corn starch, potato starch, and sodium carboxymethylcellulose. The formulation also may be presented with dibasic calcium phosphate anhydrous or sodium starch glycolate. Moreover, the formulation may be presented with lubricants, such as talc and magnesium stearate.

For parenteral administration (i.e., administration by injection through a route other than the alimentary canal), the pharmaceutical composition of the present invention may be combined with a sterile aqueous solution that may be isotonic with the blood of the subject. Such a formulation may be prepared by dissolving the pharmaceutical composition of the present invention in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampoules or vials. The formulation may be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, and sublingual.

For transdermal administration, the pharmaceutical composition of the present invention may be combined with skin penetration enhancers, such as propylene glycol, polyethylene glycol, isopropanol, ethanol, oleic acid, N-methylpyrrolidone, and the like, which increase the permeability of the skin to the pharmaceutical composition, and permit the pharmaceutical composition to penetrate through the skin and into the bloodstream. The pharmaceutical composition of the present invention may be further combined with a polymeric substance, such as ethylcellulose, methylcellulose, hydroxypropyl cellulose, ethylene/vinylacetate, polyvinyl pyrrolidone, and the like, to provide the composition in gel form, which may be dissolved in a solvent, such as methylene chloride, evaporated to the desired viscosity, and then applied to backing material to provide a patch.

The pharmaceutical composition of the present invention may comprise a pharmacological effective amount of a compound and a pharmaceutically-acceptable carrier. The pharmaceutically-acceptable carrier may be “acceptable” in the sense of being compatible with the other ingredients of the composition, and not deleterious to the recipient thereof. The pharmaceutically-acceptable carrier employed herein may be selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical composition of the present invention may be prepared by methods well-known in the pharmaceutical arts, such as, using methods disclosed in Remington's Pharmaceutical Sciences (18^{th} ed., Mack Publishing Company, Easton, Pa. (1990)). For example, the composition may be brought into association with a carrier or diluent, as a suspension or solution, such as, dissolution or suspension of the components in a vehicle, e.g., water or naturally occurring vegetable oil like sesame, coconut, peanut, or cottonseed oil or a synthetic fatty vehicle like ethyl oleate or the like. Optionally, one or more accessory ingredients (e.g., buffers, flavoring agents, surface active agents, and the like) also may be added. The choice of carrier will depend upon the route of administration of the composition. Formulations of the composition may be conveniently presented in unit dosage, or in such dosage forms, without limitation, as aerosols, capsules, elixirs, emulsions, eye drops, injections, liquid drugs, pills, powders, granules, suppositories, suspensions, syrup, tablets, or troches, which may be administered orally, topically, or by injection, including, without limitation, intravenous, intraperitoneal, subcutaneous, and intramuscular injection.

In general, the present invention comprises a method for treating a substance abuse-related condition in a subject, comprising, (a) administering to the subject an effective amount of at least one micro-dose of a opioid antagonist for at least one day; and, (b) administering to the subject a detoxifying amount of a second opioid antagonist; and optionally, (c) administering to the subject a third opioid antagonist for an extended period of time. The method may further comprise at (a) administering at least one sedative. The method comprises wherein the at least one sedative comprises antipsychotics, atypical antipsychotics, alpidem, amobarbital, antihistamines, barbiturates, benzodiazepines, chloral hydrate, chlorazepate, chlordiazepoxide, clonazepam, clonidine, diazepam, diethyl ether, dimenhydrinate, diphenhydramine, doxylamine, ethchlorvynol, flunitrazepam, gamma-hydroxybutyrate, glutethimide, herbal sedatives, imidazopyridines, kava, lorazepam, meprobamate, methaqualone, methyl trichloride, methyprylon, olanzapine, phenabarbitol, pentobarbital, promethazine, pyrazolopyrimidines, seroquel, secobarbital, tiagabine, tranquilers, zaleplon, zolpidem, a pharmaceutically acceptable salt or complex thereof, a combination thereof, and a pharmaceutical composition comprising the same. The method further comprises administering to the subject an effective amount of at least one opioid before or concurrent with step (a). For example, the at least one opioid may comprise opium, morphine, heroin, pethidine, methadone, buprenorphine, butorphanol, codeine, fentanyl, hydrocodone, hydromorphone, levorphanol, meperidine, oxycodone, pentazocine, propoxyphene, or tramadol, pharmaceutical formulations, pharmaceutical salts, or mixtures or combinations there of. The method comprises days of treatment wherein the at least one day of step (a) is about 1 to about 7 days.

The first, second and/or third opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof. The first opioid antagonist, the second opioid antagonist, and the third opioid antagonist comprise the same opioid antagonist or are different opioid antagonists.

The method comprises wherein step (c) comprises administering to the subject a controlled release pharmaceutical composition comprising the third opioid antagonist. The controlled release pharmaceutical composition releases the third opioid antagonist over a period of more than about 4 to about 24 weeks, or over a period of time of about 1 to 12 months.

A method for treating an opiate addiction in a subject comprising, (a) administering to a subject with an opiate addiction, an effective amount of a sedative on a treatment day; (b) administering to the subject an effective amount of a sedative and a micro-dose of an opioid antagonist on a treatment day; and (c) administering to the subject an effective amount of an opioid antagonist to detoxify the subject on a following day. The method may further comprise administering to the subject an effective amount of an opiate on at least one treatment day. The method further comprising repeating steps (a) and (b) so that the number of days of treatment range from about 2 days to about 20 days. The opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof. The method may further comprise administering to the subject a controlled release pharmaceutical composition comprising at least an opioid antagonist. The controlled release pharmaceutical composition may release the opioid antagonist over a period of more than about 4 to about 24 weeks, 1 to 12 months.

The foregoing description includes the best presently contemplated mode of carrying out the present invention. This description is made for the purpose of illustrating the general principles of the present inventions and should not be taken in a limiting sense. This invention is further illustrated by the following examples, which are not to be construed in any way as imposing limitations upon the scope thereof. On the contrary, it is to be clearly understood that resort may be had to various other aspects, modifications, and equivalents thereof, which, after reading the description herein, may suggest themselves to those skilled in the art without departing from the spirit of the present invention.

### EXAMPLES

### Example 1

A subject for treatment was a 25 yr old female addicted to oxycontin. The patient was screened by the intake counselor and the physician over the phone and found to be appropriate for treatment. The patient was prescribed clonidine 0.1 mg and stopped her oxycontin use on the day prior to day one. On day 1, the subject received a physical and mental evaluation, and laboratory tests were performed. Based on these results, the subject was confirmed as fit for treatment. The subject was administered 10 mg of Valium in the office along with 5 mg of Zyprexa and 100 mg of Ultram. She also received a clonidine patch that delivers 0.2 mg daily for 1 week. On day 2, she received 50 mg of Ultram in the morning, and she continued to receive her Valium, clonidine, and Zyprexa. The subject was evaluated and found to be doing well. She was given phenobarbital and Thorazine intramuscularly, and after 60 minutes, she was administered a 50 μg micro-dose of naltrexone intramuscularly. That evening, she continued to receive her Valium, clonidine and Zyprexa. On day three, the summit day, the subject returned to the office in a fasting state. The subject was lightly sedated with oral valium, and phenobarbital and Thorazine, which were both administered intravenously. While she was lightly sedated, she received intravenous administration of the 2000 μg of naloxone over a period of 5 hours. During the 5 hour administration, very small doses of naloxone were initially given (100 micrograms), and higher doses were given as she was able to tolerate them. After the subject received all of the IV Naloxone, she was given 2 mg of naltrexone intravenously. Following the detoxifying step on the summit day, a sustained release pellet comprising naltrexone was implanted in the abdominal fat of the subject.

### Example 2

A 24 year old subject for treatment was a female addicted to methadone. The subject was screened by the intake counselor and the physician over the phone and was found to be appropriate for treatment. The patient was prescribed clonidine 0.1 mg and stopped her methadone use two days prior to her first clinic visit. On day 1, the patient received a physical and mental evaluation, and laboratory tests were performed. Based on these results, the subject was confirmed as fit for treatment. The subject was administered 10 mg of Valium in the office along with 5 mg of Zyprexa and 100 mg of Ultram. She also received a clonidine patch that delivers 0.2 mg daily for 1 week. She was prescribed a sufficient quantity of Valium, Ultram, Clonidine, and Zyprexa, such that her support person was able to give her these medicines under supervision, during each of the 8 days of her detoxification. Her daily dose was about 0.4 mg of clonidine each day, along with 15 mg of Zyprexa, 250 mg of Ultram and 40 to 60 mg of Valium. On day 2, she was evaluated and found to be doing well. She was administered a 25 μg micro-dose of naltrexone intramuscularly. That evening, and each day thereafter, she continued to receive her Valium, clonidine and Zyprexa. On day three, she returned to the office and received a 50 μg micro-dose of naltrexone. She returned to the office each day, and her micro-dose of naltrexone was increased so that by day 6 she received a micro-dose of 125 μg of naltrexone. On day 7, the day before the summit day, she stopped her Ultram use after her morning dose of 50 mg. She returned to the office and, after evaluation, she received 65 mg of phenobarbital intramuscularly and 100 mg of Thorazine intramuscularly. After 60 minutes, she received a 100 μg micro-dose of naltrexone. On day 8, the summit day, she returned to the office in a fasting state. The subject was lightly sedated with oral Valium, phenobarbital and Thorazine, which were both administered intravenously. While she was lightly sedated, she received an intravenous administration of the 2000 μg of naltrexone over a period of 5 hours. Very small doses were initially given (100 μg), and higher doses were given as she was able to tolerate them. After she received all of the IV naloxone, she was given 2 mg of naltrexone intravenously. Following the detoxifying step on the summit day, a sustained release pellet comprising Naltrexone was implanted in the abdominal fat of the subject.

All patents, patent applications and references included herein are specifically incorporated by reference in their entireties.

Whereas this invention has been described in detail with particular reference to specific aspects, it will be apparent to those skilled in the art that various modifications and variations can be made in the present invention in light of the above teachings without departing from the scope or spirit of the invention. Other aspects of the invention will be apparent to those skilled in the art from consideration of the specification and practice of the invention disclosed herein. It is intended that the specification and examples be considered exemplary only, with a true scope and spirit of the invention being indicated by the following claims.

What is claimed is:

1. A method for treating a substance abuse-related condition in a subject, comprising,
(a) administering to the subject an effective amount of at least one micro-dose of a opioid antagonist for at least one day; and
(b) administering to the subject a detoxifying amount of a second opioid antagonist; and optionally,
(c) administering to the subject a third opioid antagonist for an extended period of time.

2. The method of claim 1, further comprising at (a) administering at least one sedative.

3. The method of claim 2, wherein the at least one sedative comprises antipsychotics, atypical antipsychotics, alpidem, amobarbital, antihistamines, barbiturates, benzodiazepines, chloral hydrate, chlorazepate, chlordiazepoxide, clonazepam, clonidine, diazepam, diethyl ether, dimenhydrinate, diphenhydramine, doxylamine, ethchlorvynol, flunitrazepam, gamma-hydroxybutyrate, glutethimide, herbal sedatives, imidazopyridines, kava, lorazepam, meprobamate, methaqualone, methyl trichloride, methyprylon, olanzapine, phenabarbitol, pentobarbital, promethazine, pyrazolopyrimidines, seroquel, secobarbital, tiagabine, tranquilers, zaleplon, zolpidem, a pharmaceutically acceptable salt or complex thereof, a combination thereof, and a pharmaceutical composition comprising the same

4. The method of claim 1, further comprising administering to the subject an effective amount of at least one opioid before or concurrent with step (a).

5. The method of claim 4, wherein the at least one opioid comprises opium, morphine, heroin, pethidine, methadone, buprenorphine, butorphanol, codeine, fentanyl, hydrocodone, hydromorphone, levorphanol, meperidine, oxycodone, pentazocine, propoxyphene, or tramadol, pharmaceutical formulations, pharmaceutical salts, or mixtures or combinations there of.

6. The method of claim 1, wherein the at least one day of step (a) is about 1 to about 7 days.

7. The method of claim 1, wherein the first opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof.

8. The method of claim 1, wherein the second opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof.

9. The method of claim 1, wherein the third opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof.

10. The method of claim 1, wherein step (c) comprising administering to the subject a controlled release pharmaceutical composition comprising the third opioid antagonist.

11. The method of claim 10, wherein the controlled release pharmaceutical composition releases the third opioid antagonist over a period of more than about 4 to about 24 weeks.

12. The method of claim 10, wherein the controlled release pharmaceutical composition releases the third opioid antagonist over a period of time of about 1 to about 12 months.

13. The method of claim 1, where the first opioid antagonist, the second opioid antagonist, and the third opioid antagonist comprise the same opioid antagonist.

14. The method of claim 1, where the first opioid antagonist, the second opioid antagonist, and the third opioid antagonist comprise different opioid antagonist.

15. A method for treating an opiate addiction in a subject comprising,
(a) administering to a subject with an opiate addiction, an effective amount of a sedative on a treatment day;
(b) administering to the subject an effective amount of a sedative and a micro-dose of an opioid antagonist on a treatment day; and
(c) administering to the subject an effective amount of an opioid antagonist to detoxify the subject on a following day.

16. The method of claim 15 further comprising administering to the subject an effective amount of an opiate on at least one treatment day.

17. The method of claim 15 further comprising repeating steps (a) and (b) so that the days of treatment range from about 2 to about 20.

18. The method of claim 15, wherein the opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, lofexidine, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof.

19. The method of claim 15, further comprising administering to the subject a controlled release pharmaceutical composition comprising at least an opioid antagonist.

20. The method of claim 19, wherein the controlled release pharmaceutical composition releases the at least one opioid antagonist over a period of more than about 4 to about 24 weeks.
